(19) United States
(12) Patent Application Publication
(10) Pub. No.: US 2020/0377434 A1
(43) Pub. Date: Dec. 3, 2020

(54) METHOD OF PRODUCING COMPOUND HAVING BUTADIENE SKELETON CONTAINING HYDROGEN AND FLUORINE AND/OR CHLORINE

(71) Applicants: KANTO DENKA KOGYO CO., LTD., Tokyo (JP); NATIONAL UNIVERSITY CORPORATION GUNMA UNIVERSITY, Maebashi-shi, Gunma (JP)

(72) Inventors: Hideki AMII, Maebashi-shi, Gunma (JP); Mitsuharu SHIMODA, Shibukawa, Shibukawa-shi, Gunma (JP); Yoshihiko IKETANI, Shibukawa, Shibukawa-shi, Gunma (JP); Ryo KIMURA, Shibukawa, Shibukawa-shi, Gunma (JP)

(21) Appl. No.: 16/607,187

(22) PCT Filed: Apr. 26, 2018
(86) PCT No.: PCT/JP2018/016930

§ 371 Date: Oct. 22, 2019

(30) Foreign Application Priority Data
Apr. 27, 2017 (JP) 2017-088665

(51) Int. Cl. C07C 17/269; B01J 23/72

(52) U.S. Cl. CPC C07C 17/269; B01J 23/72

(57) ABSTRACT

An object of the present invention is to provide a simple, low-cost, and industrial method of producing a compound having a polyene skeleton containing hydrogen and fluorine and/or chlorine.
A method of producing a. halogenated diene represented by formula (1): A¹A²C=CA³-CA⁴=CA⁵A⁶ [A¹, A², A⁵, and A⁶ are each independently hydrogen, fluorine, chlorine, a (perfluoro)alkyl group having 1 to 3 carbon atoms, or a (perfluoro)alkenyl group; A³ and A⁴ are each independently hydrogen, fluorine, or chlorine; at least one of A¹ to A⁶ is hydrogen; at least one of A¹ to A⁶ is fluorine or chlorine] comprises a step of subjecting the same or different halogenated olefin(s) represented by for (2): A⁷A⁸C=CA⁹X [A⁷ and A⁸ are each independently hydrogen, fluorine, chlorine, a (perfluoro)alkyl group having 1 to 3 carbon atoms, or a (perfluoro)alkenyl group; A⁹ is each independently hydrogen, fluorine, or chlorine; X is bromine or iodine] to a coupling reaction in the presence of a zero-valent metal.

### TECHNICAL FIELD

The present invention relates to a method of producing a compound having a polyene skeleton, especially butadiene skeleton, containing hydrogen and fluorine and/or chlorine.

### BACKGROUND ART

As a synthetic method for a hydrofluorocarbon (HFC) compound having a 1,3-butadiene skeleton, a coupling reaction using two metals is known as in the following formula (Non Patent Literature (NPL) 1). In this reaction, 1,1-difluoroiodoethylene, as a starting material, was mixed with an excess amount of metallic zinc, and then, the target reaction is allowed to proceed through a coupling reaction using a catalytic amount of the expensive palladium catalyst.

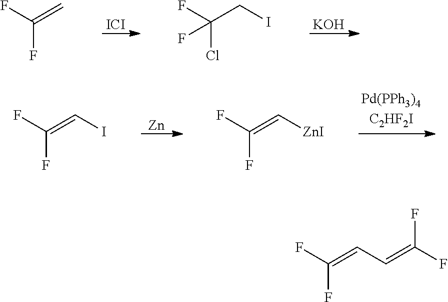

Moreover, it is known that 1,1,4,4-tetrafluoro-1,3-butadiene can be synthesized by involving a cyclization reaction between tetrafluoroethylene and acetylene as well as pyrolysis as shown in the following formula. However, this cyclization reaction requires a high temperature of 600° C. (NPL 2).

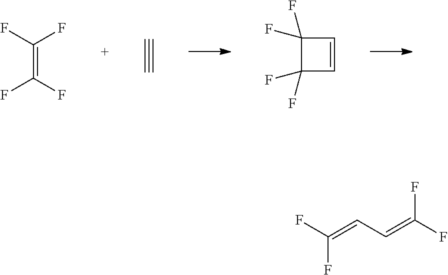

Further, as a production method for a fluorocarbon that bears no hydrogen atom bonded with a double bond, rather than for a hydrofluorocarbon, hexafluoro-1,3-butadiene is obtained by reacting 1,1,2-trifluoro-2-iodoethylene with copper powder as shown in the following formula (Patent Literature (PTL) 1).

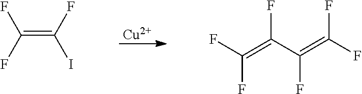

### CITATION LIST

### Patent Literature

PTL 1: Japanese Unexamined Patent Application Publication (Translation of PCT Application) No. 2008-510832

### Non Patent Literature

NPL 1: Angew. Chem. Int. Ed., 2002, 41, 296-299

NPL 2: J. Arm Chem. Soc., 1961, 83, 382-385

### SUMMARY OF INVENTION

### Technical Problem

Neither of the above-described background art is considered to be usable for mass production of hydrofluorobutadiene due to the expensive metal catalyst, high-temperature reaction conditions, reaction substrate limited to perhalogenated ethylene, or the like. Accordingly, an object of the present invention is to provide a simple, low-cost, and industrial method of producing a compound having a polyene skeleton, especially butadiene skeleton, containing hydrogen and fluorine and/or chlorine.

### Solution to Problem

The present inventors conducted intensive studies o achieve the above-mentioned object and as a result, found a high-yield, simple, low-cost, and industrial method of producing a compound having a polyene skeleton containing hydrogen and fluorine, thereby completing the present invention. Specifically, the present invention provides the following.

[1] A method of producing a halogenated diene represented by formula (1):

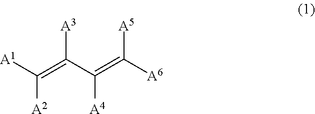

wherein: A¹, A², A⁵, and A⁶ are each independently hydrogen, fluorine, chlorine, a (perfluoro)alkyl group having 1 to 3 carbon atoms, or a (perfluoro)alkenyl group;

A³ and A⁴ are each independently hydrogen, fluorine, or chlorine; and

at least one of A¹ to A⁶ is hydrogen, and at least one of A¹ to A⁶ is fluorine or chlorine,

the method comprising a step of subjecting the same or different halogenated olefin(s) represented by formula (2) to a coupling reaction in the presence of a zero-valent metal:

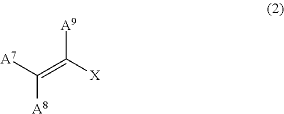

wherein: A⁷ and A⁸ are each independently hydrogen, fluorine, chlorine, a (perfluoro)alkyl group having 1 to 3 carbon atoms, or a (perfluoro)alkenyl group;

A⁹ is each independently hydrogen, fluorine, or chlorine; and

X is bromine or iodine,

[2] The method according to [1], comprising the step of subjecting the same halogenated olefin to a coupling reaction.

[3] The method according to [1], comprising the step of subjecting the different halogenated olefins to a coupling reaction.

[4] The method according to any one of [1] to [3], where at least one of the halogenated olefin(s) represented by formula (2) is 1,1-difluoro-2-iodoethylene.

[5] The method according to any one of [1] to [4], where the metal is copper.

[6] The method according to any one of [1] to [5], where the step of subjecting to a coupling reaction is performed in a solvent or in the absence of solvent

[7] The method according to [6], where the solvent is one or more selected from amide solvents.

[8] The method according to any one of [1] to [7], where a reaction temperature in the step of subjecting to a coupling reaction is within a range of 20° C. to 200° C.

### Advantageous Effects of Invention

The present invention enables high-yield, simple, low-cost, and industrial production. of the above-described halogenated diene represented by formula (1) having at least one hydrogen atom and at least one fluorine atom and/or chlorine atom.

### DESCRIPTION OF EMBODIMENTS

(Action)

The present invention is a method of producing the halogenated diene represented by formula (1) and is characterized by comprising a step of subjecting the same or different halogenated olefin(s) represented by formula (2) to a coupling reaction in the presence of a zero-valent metal.

As far as the applicants know, a reaction of coupling halogenated olefins whose double-bond carbons are bonded with one or more hydrogen atoms by using a zero-valent metal, especially zero-valent copper, without using an expensive noble metal catalyst had not been proposed. Under such circumstances, the present invention was experimentally found and is totally unpredictable to those skilled in the art.

(Reaction Substrates)

The reaction substrate(s) of the present invention is the halogenated olefin(s) represented by formula (2). In formula (2), A⁷ and A⁸ are each independently hydrogen, fluorine, chlorine, a (perfluoro)alkyl group having 1 to 3 carbon atoms, or a (perfluoro)alkenyl group; A⁹ is hydrogen, fluorine, or chlorine; and X is bromine or iodine. Two of the same or different such reaction substrate(s) are coupled at the site bonded with X, thereby forming the halogenated diene represented by formula (1). Provided that at least one of A¹ to A⁶ is hydrogen, and at least one of A¹ to A⁶ is fluorine or chlorine in formula (1), two of the same or different halogenated olefin(s) represented by formula (2) are selected for reaction substrates. Here, examples of the (perfluoro)alkyl group having 1 to 3 carbon atoms include a trifluoromethyl group, a pentafluoroethyl group, an n-heptafluoropropyl group, and a heptafluoroisopropyl group. Although the carbon number is not limited, the (perfluoro)alkenyl group is desirably selected such that the number of double bonds in product polyenes becomes 2 to 6. Specific examples include a trifluorovinyl group, a 1,2,3,4,4-pentafluoro-1,3-butadienyl group, and a 1,2,3,4,5,6,6-heptafluoro-1,3,5-hexatrienyl group.

Specific examples of the halogenated olefin(s) represented by formula (2) include 1,1-difluoroiodoethylene, 1,2-difluoroiodoethylene, 2-fluoroiodoethylene, 1-fluoroiodoethylene, iodoethylene, 1,1-difluorobromoethylene, 1,1-dichloroiodoethylene, and 1,1,2-trifluoroiodoethylene.

(Zero-valent Metals)

In the present invention, a zero-valent metal is required to be present in the reaction system as a catalyst. Examples of the metal include copper, zinc, magnesium, iron, silver, aluminum, and nickel, and copper is preferably used. To increase the surface area for reactions, the metal is preferably granular. In such a case, the particle size is preferably 10 μm to 1 mm and more preferably about 20 to 80 μm, for example. Since the metal surface is generally oxidized, the catalyst activity is low. Accordingly, before feeding to the reaction system, the metal is preferably subjected to pretreatment for removing ionized metal, such as oxides or nitrides, from the metal surface. An exemplary pretreatment includes mixing with an acid, filtering after stirring, washing with pure water and acetone, followed by heat vacuum drying, and the like.

(Reaction Conditions)

The coupling reaction of the present invention is performed by heating the halogenated olefin(s) represented by formula (2) in the presence of a zero-valent metal. The reaction temperature is preferably 20° C. to 200° C. and more preferably 100° C. to 150° C. The reaction pressure is typically atmospheric pressure, but when the reaction substrate is a gas, the reaction can be performed by placing a zero-valent metal inside a pressure-resistant reaction vessel and introducing the gas into the reaction vessel. The reaction can be terminated by lowering the reaction temperature to room temperature.

When the reaction substrate is a liquid, the coupling reaction is preferably performed in a solvent since uniform reaction is possible. Exemplary solvents include amide solvents, and specifically, DMF (N,N-dimethylformamide), NMP (N-methyl-2-pyrrolidone), and so forth may be used.

The halogenated diene of formula (1), which is the product of the present invention, may be purified by a method known in the relevant field and is commonly purified by distillation.

### EXAMPLES

(Copper Activation Method)

Copper powder was added to hydrochloric acid, mixed, suction filtered, and washed with pure water and then with acetone. The washed copper powder was subjected to heat vacuum drying at 150° C.

### Example 1

To a round-bottom flask equipped with a mechanical stirrer, a thermometer, a condenser cooled to −20° C., and a trap cooled with dry ice, copper powder (particle size of about 20 to 40 μm, 267.60 g, 4.21 mol) activated by the above-described method and DMF (198 mL) were fed and heated to 130° C. in an oil bath. To the heated solution, 1,1-difluoroiodoethylene C₂HF₂I (200.05 g, 1.05 mol) synthesized in accordance with the method described in NPL 1 was added dropwise at a rate of 1 g/min. After the end of the dropwise addition, the temperature of the condenser was set to 5° C. and the reaction solution was stirred for 3 to 4 hours. Subsequently, the oil bath temperature was raised to 150° C., and the reaction solution was stirred for 30 minutes and then cooled to room temperature. Weighing and GC analysis of the trapped gas (the amount of trapped gas: 67.5 g, GC purity: 88%) revealed the yield of the product (1,1,4,4-tetrafluorobutadiene) of 88% as a crude yield based on 1,1-difluoroiodoethylene.

### Example 2

The reaction was performed in the same manner as Example 1 except for changing the solvent from DMF (198 mL) to NMP (198 mL). The product (1,1,4,4-tetrafluorobutadiene) was obtained at crude yield of 30% based on 1,1-difluoroiodoethylene.

### Example 3

To a round-bottom flask equipped with a mechanical stirrer, a thermometer, a condenser cooled to −15° C., and a trap cooled with dry ice, copper powder (particle size of about 20 to 40 μm, 255 g, 4.0 mol) activated by the above-described method and DMF (200 mL) were fed and heated to 140° C. in an oil bath. To the heated solution, a mixture of 1,1-difluoroiodoethylene C₂HF₂I (95 g, 0.5 mol) synthesized in accordance with the method described in NPL 1 and 1,1,2-trifluoroiodoethylene C₂F₃I (104 g, 0.5 mol) was added dropwise. After the end of the dropwise addition, the temperature of the condenser was set to 15° C. and the reaction solution was stirred for 2 hours. Subsequently, the oil bath temperature was raised to 150° C., and the reaction solution was stirred for 30 minutes and then cooled to room temperature. Weighing and GC analysis of the trapped gas (the amount of trapped gas: 74 g, GC purity: 54%) revealed the yield (percentage converted from the product mole relative to 100% for 0.5 mol) of the product (1,1,2,4,4-pentafluorobutadiene) of 55% as a crude yield. Moreover, the obtained compound was identified to be 1,1,2,4,4,-pentafluorobutadiene by ¹⁹F-NMR measurement and GCMS analysis [M⁺144].

1. A method of producing a halogenated diene represented by formula (1): 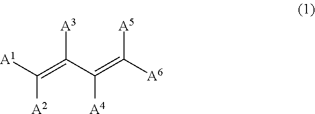
wherein: A¹, A², A⁵, and A⁶ are each independently hydrogen, fluorine, chlorine, a (perfluoro)alkyl group having 1 to 3 carbon atoms, or a (perfluoro)alkenyl group;
A³ and A⁴ are each independently hydrogen, fluorine, or chlorine; and
at least one of A¹ to A⁶ is hydrogen, and at least one of A¹ to A⁶ is fluorine or chlorine,
the method comprising a step of subjecting the same or different halogenated olefin(s) represented by formula (2) to a coupling reaction in the presence of a zero-valent metal: 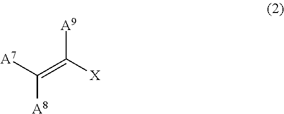
wherein: A⁷ and A⁸ are each independently hydrogen, fluorine, chlorine, a (perfluoro)alkyl group having 1 to 3 carbon atoms, or a (perfluoro)alkenyl group;
A⁹ is each independently hydrogen, fluorine, or chlorine; and
X is bromine or iodine.

2. The method according to claim 1, comprising the step of subjecting the same halogenated olefin to a coupling reaction.

3. The method according to claim 1, comprising the step of subjecting the different halogenated olefins to a coupling reaction.

4. The method according to claim 1, wherein at least one of the halogenated olefin(s) represented by formula (2) is 1,1-difluoro-2-iodoethylene.

5. The method according to claim 1, wherein the metal is copper.

6. The method according to claim 1, wherein the step of subjecting to a coupling reaction is performed in a solvent or in the absence of solvent.

7. The method according to claim 6, wherein the solvent is one or more selected from amide solvents.

8. The method according to claim 1, wherein a reaction temperature in the step of subjecting to a coupling reaction is within a range of 20° C. to 200° C.
